(19) United States
(12) Patent Application Publication
(10) Pub. No.: US 2018/0125138 A1
(43) Pub. Date: May 10, 2018

(54) SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT

(71) Applicant: Brian Quaglia, Rehoboth, MA (US)

(72) Inventor: Brian Quaglia, Rehoboth, MA (US)

(21) Appl. No.: 15/713,636

(22) Filed: Sep. 23, 2017

Related U.S. Application Data
(63) Continuation-in-part of application No. 15/408,307, filed on Jan. 17, 2017.
Application No. 15/408,307 is a continuation-in-part of application No. 15/600,790, filed on May 22, 2017, now abandoned.
(60) Provisional application No. 62/403,157, filed on Oct. 2, 2016.

(51) Int. Cl. A41G 7/00; A61F 9/04; A41D 1/00; A41D 31/00; B63C 11/20; A63J 7/00; A41D 27/08; A63B 33/00; A63B 69/00; A63B 71/06

(52) U.S. Cl. CPC A41G 7/00; A61F 9/04; A41D 1/002; A41D 31/00; B63C 11/205; A63J 7/005; A63B 2207/02; A63B 33/002; A63B 69/00; A63B 71/0622; A41D 2400/26; A63B 2208/03; A63B 2071/0625; A41D 27/085

(57) ABSTRACT

A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT providing under water vision with a water-tight sealing portion or mechanism securing the eyepieces around the eye area of a user during use of the goggles and a material or fabric costume mask element conducive to being worn by a child or adult and to even help in aiding the process of learning to swim.

### CLAIM OF PRIORITY

This non-provisional application is a continuation-in-part and takes priority to and the benefit of non-provisional application number 15/408,307 filed on Jan. 17, 2017 and this non-provisional application is a continuation-in-part and takes priority to and the benefit of non-provisional application number 15/600,790 filed on May 22, 2017 and takes priority to provisional application 62/403,157 filed on Oct. 2, 2016, which both are applications are incorporated herein in both of their entireties by this reference.

### FIELD OF INVENTION

The invention is in the field of swimming eyewear devices, more particularly a swim goggles headwear and costume system with a mask, a costume or costume mask element, and an integrated goggles, eyewear, and/or lenses element.

### BACKGROUND OF INVENTION

Swimming goggles and lenses have been around many years. Everyone is familiar with the basic lap swimming simple clear plastic shell cups which are secured around your head usually using a rubber band type material that is attached to the cups and the cups create a vacuum and chamber for the eyes and usually are attached to each other with a plastic piece fitting over the nose. There have been lens shaped cups attached to fabric to secure the eyes to a fabric mask for marc-o-polo type games where the device is not swim goggles or lenses as you can't see as the lens holder shaped device or devices portion only functions to secure the mask in place on a person's face and do not allow for opening eyes to see. The purpose of these products is not to see anything.

A child (or even an adult) enthusiastically wants to engage in fun activity and fun role play activity, with outcome of a positive experience.

Swim Garments and Outfits can be styled as Costumes for a child to wear while learning to swim or for children and others to wear during swimming, water play, and just play or dress up, and in general can be used for dress-up and role playing to enhance the swimming experience. Garments can also include floatation as part of a swimming Costume.

The present invention enhances the play value of swimming eyewear. People both young and old love masks whether for a masquerade ball, or Halloween, or other occasion, or simply role play fun, which can be styled so many ways from bright colored patterns to scary monster looks. A costume swim mask or costume element can include printing to enhance the authenticity of the Costume or Character Branded with printed Characters, logos, etc. These would be what we are calling Two-dimensional Costume Swim Masks There can be objects also attached to the fabric or three-dimensional aspects of the fabric itself.

See definitions below for:

### Character Branded Mask

### Costume

### Costume Mask

### Costume Swim Mask

Printed Costume mask

### Two-dimensional Costume Mask and Two-dimensional Costume Swim Mask

### Two-Dimensional Printed Costume Mask

### Three-dimensional Costume Mask

### Three-dimensional Swim Costume Mask

Three-dimensional Swimming Costume mask.

The present invention incorporates under water vision with an integrated Two or Three-dimensional Material and is at least one of a two-dimensional Swim Costume Mask, two-dimensional Swimming Costume mask, Three-dimensional Swim Costume Mask, and/or Three-dimensional Swimming Costume mask. This Swim Mask is fun for a child or adult to wear. This Costume Mask can also aid in teaching a child or adult to swim as well, make the child less fearful of water, and thus more inclined to enter the water and learn to swim.

### SUMMARY OF INVENTION

A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT comprising:

at least one of: goggles; eyewear; and/or lenses element;
said lenses element, if present, is comprised of a pair of connected or unconnected eyepieces;
any of said lenses, goggles, or eyewear elements, if present, are adapted to provide under water vision; a water-tight sealing portion, device, part, or mechanism securing the goggles, eyewear, and/or lenses element, that is present, and/or eyepieces around the eye area of a user during use of the said swim goggles headwear and costume system;
and a costume mask having:
a material or fabric mask or mask element, having a pair of eyeholes;
and at least one costume or costume element, in whole or in part.

The invention may include in at least one embodiment at least one of the following:

sun protection; sun protection that is officially tested for UV protection; all-day sun protection; rash protection; the invention or any part thereof partially covers the wearer's face; the invention, or the said mask, or the said costume or said costume swim mask partially covers the wearer's face leaving the lower part of his face free; wherein the invention, or the said mask, or the said costume or costume swim mask fully covers the wearer's face; wherein the invention, or the said mask, or the said costume or costume swim mask extends outward past the wearer's face extending outward in any combination, size, shape, length, depth, thickness, and/or degree to the: left; right; up; down; out in front; around; and/or behind; to further enhance the costume aspect and/or add more attachability; wherein the invention, or the said mask, or the said costume or costume swim mask fully covers the wearer's face having the lower, upper, top, and/or back part of his/her face covered; wherein the said mask, costume or costume swim mask extends outward past the wearer's face and/or head and/or body extending outward in any combination, size, shape, length, depth, thickness, and/or degree to the: left; right; up; down; out in front; around; and/or behind; wherein the said mask, costume or costume swim mask extends outward past the wearer's face and/or head and/or body extending outward in any combination, size, shape, length, depth, thickness, and/or degree to the: left; right; up; down; out in front; around; and/or behind; to further enhance the costume aspect and/or add more attachability; wherein the invention or the said mask, said costume or said costume swim mask partially covers any of the wearer's face, head, and/or body in any combination, size, shape, length, depth, thickness, and/or degree; wherein the invention or the said mask, said costume, or said costume swim mask fully covers any of the wearer's face, head, and/or body in any combination, size, shape, length, depth, thickness, and/or degree; wherein if said eyepieces are present and are connected further comprising a connector device to connect said eyepieces; the invention additionally comprises at least one band or strap for encircling the wearer's head, and/or attaching by means of attaching device to said costume swim mask and/or said mask, said costume, said goggles, eyewear, and/or lenses elements, for holding the said costume swim goggles securely in place on the wearer; wherein the said at least one band or strap is an elastic band; wherein said elastic band passes through holes in the mask; the invention additionally comprising at least one three-dimensional costume component; the invention further comprising of an inner mesh liner to keep said costume mask secure on the wearer; the invention having a front side and a back side; the invention additionally comprising at least one of: an inner mesh liner to keep the invention secure on the wearer; at least one buoyant floatation element contained within and/or between said at least one front layer and said at least one back layer; said at least one back layer and/or said at least one front layer attaching to said at least one flotation attachment area; at least one attachment and/or securing strap; wherein said costume swim mask is made from at least one of, in whole or in part, a fast-drying material such as nylon, spandex, rayon, synthetic material, natural material, hard plastic, sequins, waterproof and/or water-resistant materials, in whole or in part, athletic garment material that is built from at least one of the following materials of spandex, nylon, polyester, neoprene, and/or lycra (Four-Way Stretch Lycra enhances overall flexibility and comfort), in part or in whole, to ensure durability, and thus the mask is lightweight, quick drying, and flexible naturally antibacterial and water wicking; and/or including said protection material for the wearer against rashes caused by abrasion, or by sunburn from extended exposure to the sun; has said light coverage in warm to extreme summer temperatures; and/or is made from at least one of in whole or in part a fast drying material in part or in whole such as nylon, spandex, rayon, synthetic material, natural material, hard plastic, sequins, waterproof and/or water resistant materials, in whole or in part; the invention further comprising of or incorporating rigid waterproof or water resistant elements and/or fake bones and/or components to provide skeletal structure to flexible material, in whole or in part; the invention further comprising at least one of: accessories, gloves, webbed gloves, at least one hat, footwear, a snorkel, periscope, tentacles, body parts, at least one noise or sound making device; the invention further comprising of at least one auxiliary waterproof or water resistant container and or designated volume of space to house electronics and/or other objects; said electronics is at least one of: a sound module producing speech and/or music and/or sound effects; a lighting system producing at least one pattern of lights; a radio receiver; a walkie-talkie and/or at least two way communication system; a smart device and/or smart phone; the invention is further styled as at least one face and/or other part/portion of at least one character, cartoon character, mermaid, superhero, animal, princess, fairy, elf, goblin, witch, wizard, body part, likeness, image, celebrity image, photograph, thing, object, evening wear.

The invention may include a three-stage method of learning to swim that incorporates the wearing of A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT comprising the following steps:

Swim level 1, which is an introduction to the water;
Swim level 2, which is learning to swim;
Swim level 3, which is developing greater swim skill and confidence.
The invention may include a method of learning to swim that incorporates the wearing of:

A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT having at least one of the incorporation of floatation or flotation not incorporated therein while inducing a child to engage in a fun play pattern involving one or more of the following: fantasy role-playing, splashing, interacting with other swimmers of varying skill levels, humor, testing limits of interaction with water, gaining confidence and developing trust of water, learning to be comfortable with the aquatic environment.

The invention may include wherein said SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT is used for dress-up and role-play or for leisure swim and water-play other than swim training.

The invention may include that the said costume swim mask is antibacterial and/or water wicking.

The invention may be waterproof and/or water resistant.

The invention may give protection against jelly fish stings.

The invention may help prevent irritation caused by rapid impact with surface water and waves as well.

The invention may additionally comprising at least one depth component on at least one portion of the costume mask or to any component of the invention providing for a higher level of Character authenticity by the utilizing or adding of three dimensional materials either integrated or associated with or attached to and therefore significantly improving upon the role play and dress up experience of the wearer the invention incorporating texture, fabric, a greater involvement of the sense of feel so that more senses are used other than vision.

The invention may be used for dress-up and role-play or for leisure swim, watersport, and water-play.

The invention may include a flotation element or device.

Although preferred embodiments of the present invention have been described herein it will be understood by those skilled in the art that the present invention should not be limited to the described preferred embodiments. Rather, various changes and modifications can be made within the spirit and scope of the present invention.

All the material in this patent document issue subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BRIEF DESCRIPTION OF DRAWINGS

FIG. 1 is a drawing that depicts back view of one embodiment of the costume mask invention. Includes lenses and eye cup, back of the mask portion, extended costume elements on both sides and closure device.

FIG. 2 is a drawing that depicts back view of one embodiment of the costume mask invention. Includes lenses and eye cup, nose bridge, back of the mask portion, extended costume elements on both sides and closure device.

FIG. 3 is a drawing that depicts invention with elastic band attaching on both sides.

FIG. 4 depicts the front of the mask.

FIG. 5 depicts the components numbered from at least one embodiment.

FIG. 6 depicts one embodiment of the invention worn on user's face.

FIGS. 7 and 8 below are renderings of one of two eyepieces.

FIG. 9 depicts lens attached to eyepiece.

FIG. 10 is an assembly diagram for at least one embodiment.

FIG. 11 depicts the invention completely covering user's head. Front ¾ View.

FIG. 12 depicts the invention completely covering user's head. Side/Back View.

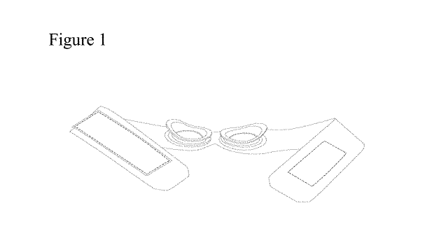

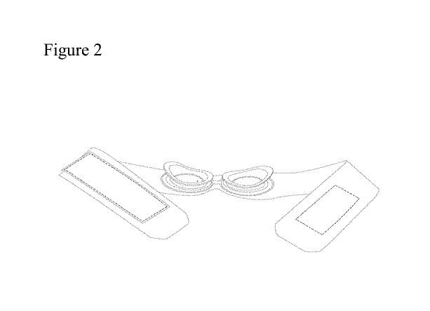

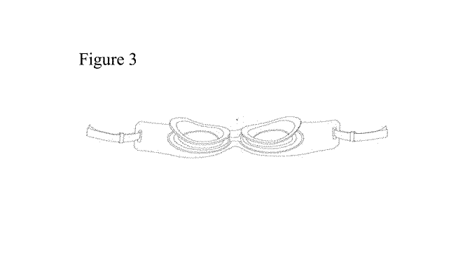

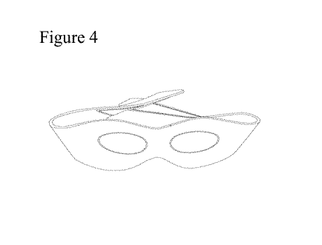

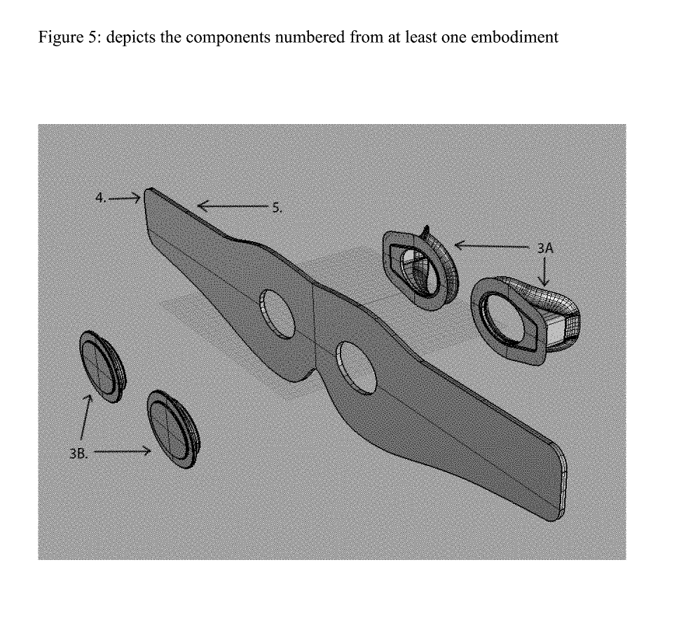

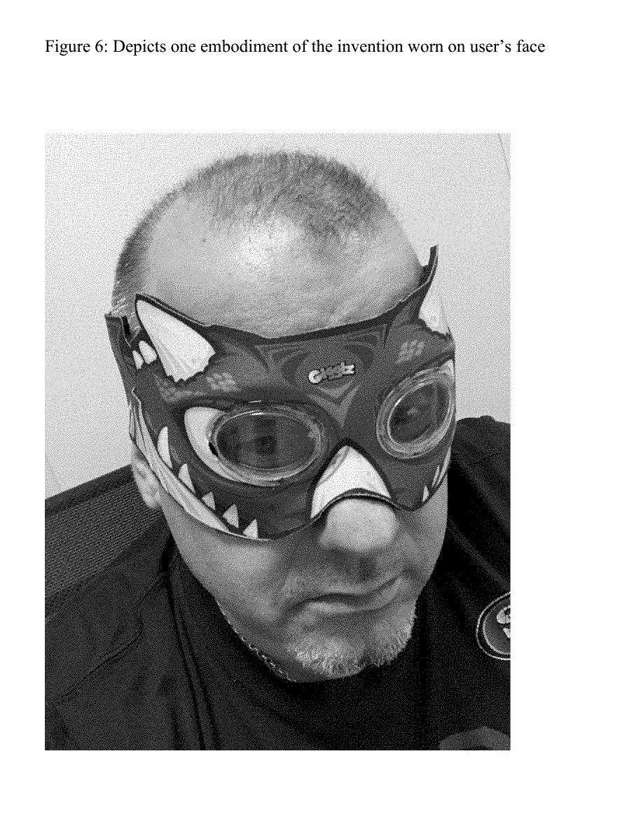

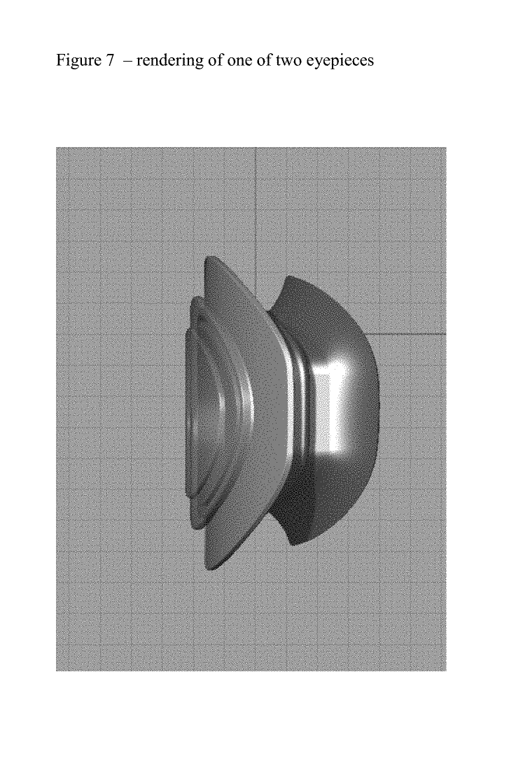

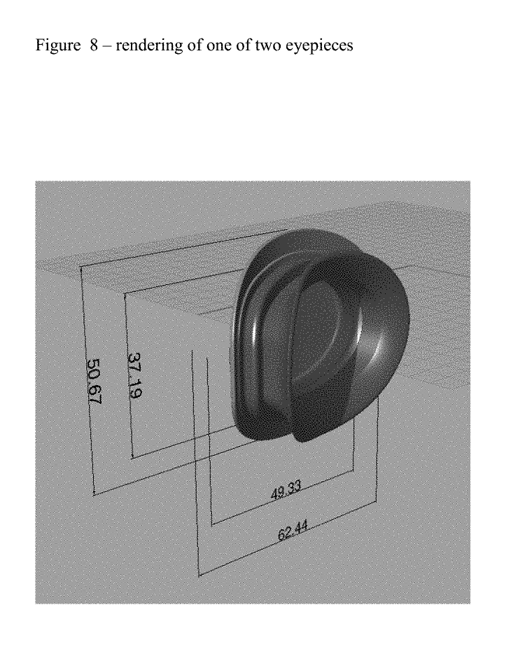

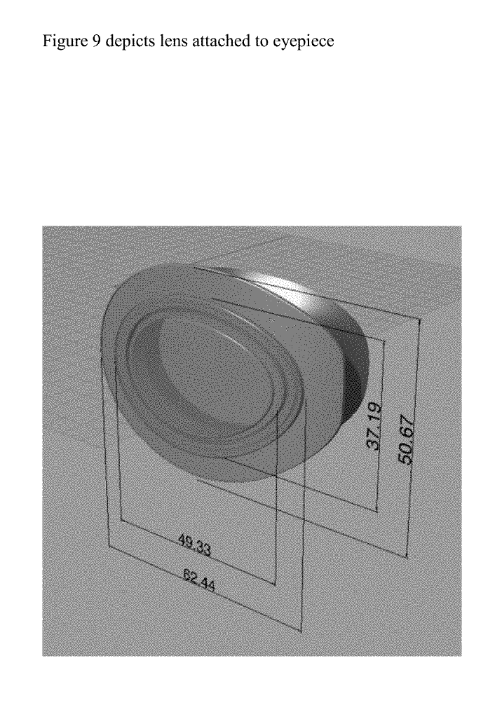

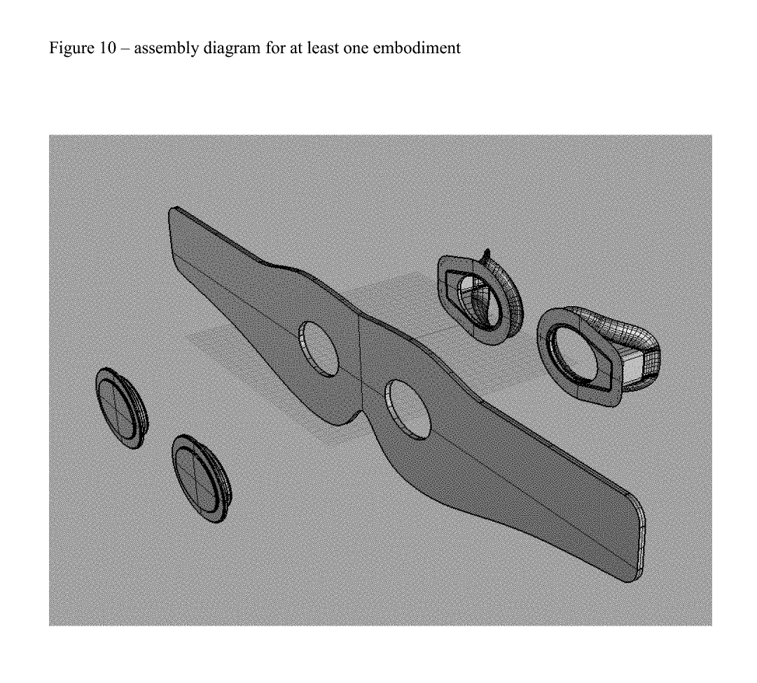

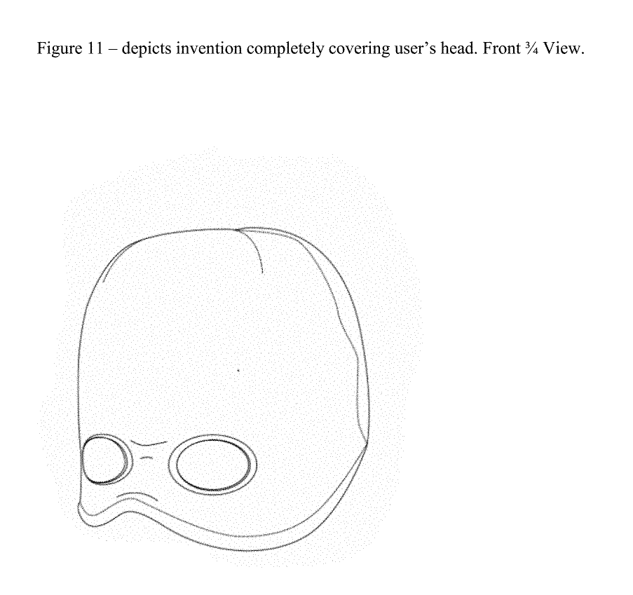

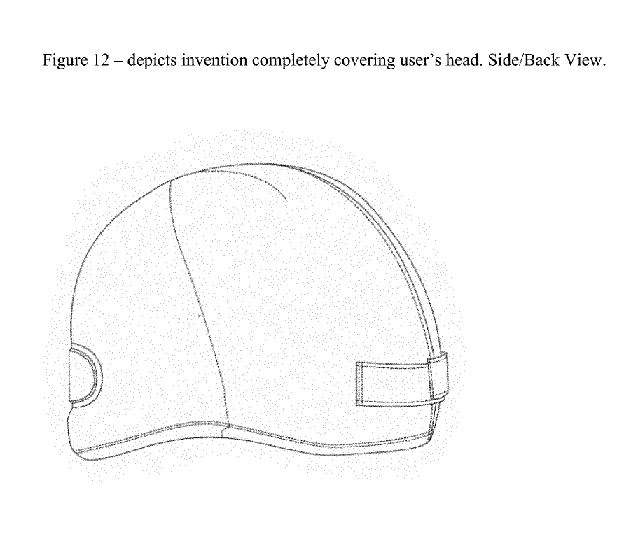

### Some of the Components

1 a swim goggles headwear system with costume mask element and integrated goggles or lens element
2 costume mask element
3 integrated goggles and/or lenses elements
3a eyepiece
3b lenses
3c nose bridge
4 front of mask
5 back of mask
6 eyewear connector
7 elastic band
8 elastic strap
9 extended costume mask sides
20 Three-dimensional costume mask

### DEFINITIONS

Outfit: is a set of clothing that goes well together.

Garment: is an item of clothing.

Character Branded Mask: is an Mask that has in at least one location an image or motif of a Character.

Character Branded: is a Character's image printed or integrated on or in a product.

Costume: is something you wear to make yourself look like something or someone and/or if you're dressing up to affect a personality or you are playing a role. A costume can also be a reproduction of a Character incorporating a level of authenticity either within or upon a Garment or Outfit or a Garment or Outfit with at least one accessory. A costume can have variable levels of authenticity depending on context and usage.

Costume Mask: is something you wear on and cover, in part or in whole, your face and/or head to make yourself look like something or someone and/or if you're dressing up to affect a personality or you are playing a role. A Costume Mask can also be a reproduction of a Character or Character part (Like face, eyes, nose, etc.) incorporating a level of authenticity either within, and/or attached to, and/or upon the mask. A costume mask can have variable levels of authenticity depending on context and usage, as well as, attachments and/or accessories if any.

Costume Swim Mask: A Costume Mask designed to include swimming, water, and water play use which may incorporate fabric and materials that are in whole or in part waterproof or water resistant and includes eyewear elements such as eyewear, Goggles or lenses.

Printed Costume mask: is a Two-dimensional Costume Mask with printing.

Two-dimensional Costume Mask: A Costume Mask made from material with no attention to any portion of the Costume Mask that touches human skin being thicker than one or more layers of fabric.

Two-Dimensional Printed Costume Mask: at least one Mask transformed to a two-dimensional Character Costume Mask by the printing forming the image or images necessary to create a level of authenticity together forming the Costume Mask.

Character: is a known or unknown animated image, a cartoon or comic image, a living or deceased celebrity, a superhero, a monster, an alien, a fictional being, a real or imaginary animal, person, place, or thing.

Printing: in the context of this invention is printing on fabric, embossing, silk-screening or other method of applying an image on material or fabric used in a Mask.

Three-dimensional Costume Mask: everything a Two-dimensional Costume is with the addition of at least one depth component to at least one portion of the Mask, and/or attached and/or associated with or to the mask. A Three-dimensional Costume Mask is a Costume Mask that provides for a higher level of Character authenticity by the utilizing or adding of three dimensional materials either integrated or associated with or attached to. A three-dimensional Costume Mask significantly improves upon the role play and dress up experience incorporating texture, fabric, a greater involvement of the sense of feel so that more senses are used other than vision.

Two-dimensional Swim Costume Mask—Is a Two-dimensional Costume Mask and System designed to include swimming, water, and water play use which may incorporate fabric and materials that are in whole or in part waterproof or water resistant and includes eyewear elements such as Goggles or lenses.

Three-dimensional Swim Costume Mask: Is a Three-dimensional Costume Mask designed to include swimming, water, and water play use which may incorporate fabric and materials that are in whole or in part waterproof or water resistant and includes a goggles, eyewear, or lenses element for swimming and underwater vision that creates a vacuum for eyes so as not to get eyes wet underwater or splashing, Three-dimensional Swim Costume mask is also sometimes called a Three-dimensional Swimming Costume mask.

Three-dimensional Swim Costume Mask and System: Is a Three-dimensional Costume Mask and System designed to include swimming, water, and water play use which may incorporate fabric and materials that are in whole or in part waterproof or water resistant and includes eyewear elements such as Goggles or lenses.

A floaty (or floaties) is a floatation toy or device used in water, a pool and/or ocean, and this can be a tube or toroid shape filled with air, a Character in the shape of an animal or other creature, object, or icon, an air mattress, a dry bag or bladder, a buoyant tube or object that can provide extra buoyancy to a person when held, laid upon, or interfaced with. A floaty can be made from a shell that contains air or a material that contains sufficient air incorporated within or is of sufficiently low density as to provide buoyancy.

As used herein, the term “and/or,” when used in a list of two or more items, means that any one of the listed items can be employed by itself, or any combination of two or more of the listed items can be employed. For example, if a device is described as containing components A, B, and/or C, the composition can contain A alone; B alone; C alone; A and B in combination; A and C in combination; B and C in combination; or A, B, and C in combination.

1. A swim goggles headwear and costume system, including a costume swim mask, having a mask element attached to at least one costume element, and at least one integrated goggles, eyewear, and/or lenses element comprising:
at least one of: goggles; eyewear; and/or lenses element;
said lenses element, if present, is comprised of a pair of connected or unconnected eyepieces;
any of said lenses, goggles, or eyewear elements, if present, are adapted to provide underwater vision;
a water-tight sealing portion, device, part, or mechanism securing the goggles, eyewear, and/or lenses element, that is present, and/or eyepieces around the eye area of a user during use of the said swim goggles headwear and costume system; and
a costume mask having:
a material or fabric mask or mask element, having a pair of eyeholes; and
at least one costume or costume element, in whole or in part.

2. The invention of claim 1 wherein the said swim goggles, eyewear, or lenses element, if present, having sun protection.

3. The invention of claim 1 wherein the said swim goggles, eyewear, or lenses element, if present, includes reliable sun protection that is officially tested for UV protection.

4. The invention of claim 2 wherein the said sun protection is all-day sun protection.

5. The invention of claim 1 wherein the said costume swim mask incorporates rash protection.

6. The invention of claim 1 wherein the invention, or the said mask, or the said costume or said costume swim mask partially covers the wearer's face.

7. The invention of claim 1 wherein the invention, or the said mask, or the said costume or said costume swim mask partially covers the wearer's face leaving the lower part of his face free.

8. The invention of claim 1 wherein the invention, or the said mask, or the said costume or costume swim mask fully covers the wearer's face.

9. The invention of claim 1 wherein the invention, or the said mask, or the said costume or costume swim mask extends outward past the wearer's face expending outward in any combination, size, shape, length, depth, thickness, and/or degree to the: left; right; up; down; out in front; around; and/or behind; to further enhance the costume aspect and/or add more attachability.

10. The invention of claim 1 wherein the invention, or the said mask, or the said costume or costume swim mask fully covers the wearer's face having the lower, upper, top, and/or back part of his/her face covered.

11. The invention of claim 1 wherein the said mask, costume or costume swim mask extends outward past the wearer's face and/or head and/or body extending outward in any combination, size, shape, length, depth, thickness, and/or degree to the: left; right; up; down; out in front; around; and/or behind.

12. The invention of claim 1 wherein the said mask, costume or costume swim mask extends outward past the wearer's face and/or head and/or body extending outward in any combination, size, shape, length, depth, thickness, and/or degree to the: left; right; up; down; out in front; around; and/or behind; to further enhance the costume aspect and/or add more attachability.

13. The invention of claim 1 wherein the invention or the said mask, said costume or said costume swim mask partially covers any of the wearer's face, head, and/or body in any combination, size, shape, length, depth, thickness, and/or degree.

14. The invention of claim 1 wherein the invention or the said mask, said costume, or said costume swim mask fully covers any of the wearer's face, head, and/or body in any combination, size, shape, length, depth, thickness, and/or degree,

15. The invention of claim 1 wherein if said eyepieces are present and are connected further comprising a connector device to connect said eyepieces.

16. The invention of claim 1 further comprising at least one band or strap for encircling the wearer's head, and/or attaching by means of attaching device to said costume swim mask and/or said mask, said costume, said goggles, eyewear, and/or lenses elements, for holding the said costume swim goggles securely in place on the wearer.

17. The invention of claim 16 wherein the said at least one band or strap is an elastic band.

18. The invention of claim 17 wherein said elastic band passes through holes in the mask.

19. The invention of claim 1 further comprising at least one three-dimensional costume component.

20. The invention of claim 1 further comprising of an inner mesh liner to keep said costume mask secure on the wearer.

21. The invention of claim 1 having a front side and a back side.

22. The invention of claim 1 comprising at least one of: an inner mesh liner to keep the invention secure on the wearer; at least one buoyant floatation element contained within and/or between said at least one front layer and said at least one back layer; said at least one back layer and/or said at least one front layer attaching to said at least one flotation attachment area; at least one attachment and/or securing strap.

23. The invention of claim 1 wherein said costume swim mask is made from at least one of, in whole or in part, a fast-drying material such as nylon, spandex, rayon, synthetic material, natural material, hard plastic, sequins, waterproof and/or water-resistant materials, in whole or in part, athletic garment material that is built from at least one of the following materials of spandex, nylon, polyester, neoprene, and/or lycra (Four-Way Stretch Lycra enhances overall flexibility and comfort), in part or in whole, to ensure durability, and thus the mask is lightweight, quick drying, and flexible naturally antibacterial and water wicking; and/or including said protection material for the wearer against rashes caused by abrasion, or by sunburn from extended exposure to the sun; has said light coverage in warm to extreme summer temperatures; and/or is made from at least one of in whole or in part a fast drying material in part or in whole such as nylon, spandex, rayon, synthetic material, natural material, hard plastic, sequins, waterproof and/or water resistant materials, in whole or in part.

24. The invention of claim 1 further comprising of or incorporating rigid waterproof or water resistant elements and/or fake bones and/or components to provide skeletal structure to flexible material, in whole or in part.

25. The invention of claim 1 further comprising at least one of: accessories, gloves, webbed gloves, at least one hat, footwear, a snorkel, periscope, tentacles, body parts, at least one noise or sound making device.

26. The invention of claim 1 further comprising of at least one auxiliary waterproof or water resistant container and or designated volume of space to house electronics and/or other objects.

27. The invention of claim 26 wherein said electronics is at least one of: a sound module producing speech and/or music and/or sound effects; a lighting system producing at least one pattern of lights; a radio receiver; a walkie-talkie and/or at least two way communication system; a smart device and/or smart phone.

28. The invention of claim 1 is further styled as at least one face and/or other part/portion of at least one character, cartoon character, mermaid, superhero, animal, princess, fairy, elf, goblin, witch, wizard, body part, likeness, image, celebrity image, photograph, thing, object, evening wear.

29. A three-stage method of learning to swim that incorporates the wearing of A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT comprising the following steps:
Swim level 1, which is an introduction to the water;
Swim level 2, which is learning to swim;
Swim level 3, which is developing greater swim skill and confidence.

30. A method of learning to swim that incorporates the wearing of:
A SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT having at least one of the incorporation of floatation or flotation not incorporated therein while inducing a child to engage in a fun play pattern involving one or more of the following: fantasy role-playing, splashing, interacting with other swimmers of varying skill levels, humor, testing limits of interaction with water, gaining confidence and developing trust of water, learning to be comfortable with the aquatic environment.

31. The invention of claim 1 wherein said SWIM GOGGLES HEADWEAR AND COSTUME SYSTEM WITH A COSTUME SWIM MASK, HAVING A MASK ELEMENT ATTACHED TO AT LEAST ONE COSTUME ELEMENT, AND AT LEAST ONE INTEGRATED GOGGLES, EYEWEAR, AND/OR LENSES ELEMENT is used for dress-up and role-play or for leisure swim and water-play other than swim training.

32. The invention of claim 1 whereby said costume mask is antibacterial and/or water wicking.

33. The invention of claim 1 is waterproof and/or water resistant.

34. The invention of claim 1 gives protection against jelly fish stings.

35. The invention of claim 1 helps prevent irritation caused by rapid impact with surface water and waves as well.

36. The invention of claim 1 additionally comprising at least one depth component on at least one portion of the costume mask or to any component of the invention providing for a higher level of Character authenticity by the utilizing or adding of three dimensional materials either integrated or associated with or attached to and therefore significantly improving upon the role play and dress up experience of the wearer the invention incorporating texture, fabric, a greater involvement of the sense of feel so that more senses are used other than vision.

37. The invention of claim 1 wherein said invention is used for dress-up and role-play or for leisure swim, watersport, and water-play.

38. The invention of claim 1 includes a flotation element or device.
